(19) United States
(12) Patent Application Publication
(10) Pub. No.: US 2012/0101344 A1
(43) Pub. Date: Apr. 26, 2012

(54) NON-CONTACT SYSTEM AND METHOD FOR MONITORING A PHYSIOLOGICAL CONDITION

(76) Inventors: CANDIDA DESJARDINS, Cleveland Heights, OH (US); Marco Costa, Cleveland, OH (US); Lynn Antonelli, Cranston, RI (US)

(21) Appl. No.: 12/912,286

(22) Filed: Oct. 26, 2010

(51) Int. Cl. A61B 5/00

(52) U.S. Cl. 600/300

(57) ABSTRACT

Systems and methods are disclosed for measuring a physiological signal of a patient without having to contact the patient. A system can include a laser configured to provide an optical beam to a surface associated with a patient. A detector receives light from the surface in response to the optical beam. A controller is configured to generate velocity data representing a velocity of the surface based on the detected interference of the optical beam and the reflected light. A processor computes a corresponding displacement waveform representing displacement of the surface based on the velocity waveform, the displacement waveform representing the physiological condition.

### GOVERNMENT INTEREST

The invention described herein may be manufactured and used by or for the Government of the United States of America for governmental purposes without the payment of any royalties thereon or therefore.

### TECHNICAL FIELD

The present invention relates generally to a system and method for optically monitoring physiological signals without having to contact the patient.

### BACKGROUND

Various devices have been developed to measure physiological conditions such as blood pressure, respiration, heart rate and the like. Many such tools presently used for monitoring blood pressure are less than optimal, and it is taken for granted that the standard sphygmomanometer is a sufficient technology. The sphygmomanometer technology is the standard non-invasive, contact method of measuring blood pressure, where a sphygmomanometer cuff is wrapped around the patient's arm above the elbow. The cuff is manually inflated in order to occlude the brachial artery, and a sensor (typically a stethoscope) is used to hear the Korotkoff sounds, corresponding to the systolic and diastolic end-points and their associated pressure values. This common method provides only systolic and diastolic pressure values for a moment in time. The technology is also limited to measuring pressures in distal arteries in the patient's periphery.

Other non-invasive methods for continuously monitoring blood pressure that by definition do not require insertion of sensors into an artery have also been developed within the last few decades. While such methods may be considered non-invasive, contact with the patient is still required, such that these methods are not suitable for certain types of applications.

Other methods and devices have been developed to provide non-contact means for measuring other physiological conditions, such as blood flow velocity, blood oxygen saturation, and the like. However, such techniques are often complicated to set up and have not been able to provide sufficient accuracy or definition of the timing of the blood pressure waveform so as to be of any significant benefit in analysis of the cardiac cycle beyond very roughly indicating basic features, such as the heart rate.

### BRIEF DESCRIPTION OF THE DRAWINGS

FIG. 1 is an example of a system that can be utilized for monitoring a physiological signal of a patient in a non-contact manner.

FIG. 2 is a block diagram depicting an example system for monitoring one or more physiological signal of a patient.

FIG. 3 is an example of a graph depicting a skin velocity waveform with respect to time.

FIG. 4 is an example of a graph depicting skin velocity and skin displacement waveforms with respect to time.

FIG. 5 is an example of a graph depicting a skin displacement waveform relative to an arterial blood pressure waveform.

FIG. 6 is an example of a graph depicting a qualitative comparison of skin displacement waveform relative to an arterial blood pressure waveform for a given patient.

### DETAILED DESCRIPTION

Systems and methods are disclosed for optically monitoring cardiac physiological signals of a patient without having to contact the patient. The systems and methods can be utilized to provide continuous information concerning one or more physiological signal of the patient, such as relating to the cardiac health of the patient. The data obtained from the system can be constantly displayed and analyzed to provide detailed information regarding the timing characteristics of the blood pressure waveform and other related cardiac health information.

It will be understood that as the pulsatile blood flow travels through the arteries, it causes expansion and contraction of these arteries and therefore change in the diameter occurs. Thus, measuring the motion (velocity and/or displacement) of the skin above an artery, due to the arterial expansion and contraction, such as shown and described herein, can provide a measure of the arterial pulse waveform. Additional processing of the velocity and/or displacement waveforms can provide additional information about other related physiological signals (e.g., respiration, heart rate and the like).

Since the systems and methods disclosed herein can be implemented in a completely non-contact manner, it has a variety of applications in a wide patient population, and would be particularly useful for burn, trauma, and neonatal patients, where standard sphygmomanometers are not necessarily applicable. The monitoring system could also be utilized on general hospital floors and in the outpatient setting as well as for triage on the battlefield. Additionally, the system is capable of taking measurements on several arteries in the body, including, but not limited to, the carotid, facial, temporal, brachial, radial, femoral, popliteal, posterior tibialis, and dorsalis pedis arteries.

Traditionally, blood pressure waveforms are obtained invasively by inserting a catheter with a pressure sensor tip into an artery, such as the femoral or radial artery. This conventionally invasive measurement is necessary to monitor the cardiovascular health of the patient, but presents complications such as thrombosis and hematomas, as well as increasing the risk of infection. Thus, the blood pressure waveform is usually monitored only in intensive care units or catheterization labs. The non-invasive and non-contact monitoring approach to determine the blood pressure waveform is therefore an appealing alternative, lowering patient distress and the risk of life-threatening complications.

A non-contact device can also provide a means for measuring the blood pressure waveforms in the ambulatory patient population, where less informative static sphygmomanometer pressure readings are used in place of intra-arterial catheters. Many patients are advised to consistently monitor their own blood pressure, however in-home measurements are typically inaccurate and require extensive patient training. There is a natural variation of blood pressure throughout the course of a day, even within just a few minutes, so a single blood pressure reading at one particular time of the day can provide a misrepresentation of the patient's cardiovascular status. The use of continuous blood pressure monitoring can be helpful for patients with highly variable blood pressures, such as renal dialysis patients; however, an automated blood pressure cuff inflating throughout the day can cause significant patient distress, particularly while the patient is asleep, and possible limb trauma.

An example embodiment of the invention can be implemented as a small, compact apparatus that can quickly be utilized. For example, the apparatus can be self-contained with an internal power source (e.g., battery powered) for use even in locations without a power source. The apparatus is easy to use, and does not require elaborate adjustments of lasers, so that minimal training would be required before using the device. Thus, an example apparatus can be implemented to provide a versatile and efficient non-contact means to measure blood pressure related signals.

The use of lasers to remotely detect the blood pressure waveform and heart rate without (or without) contact can provide a powerful diagnostic tool. The analysis can be utilized to provide diagnostic information or information based on which a medical professional can render a diagnosis. For instance, analysis of the derived blood pressure waveform can be utilized to detect different forms of cardiac disease, such as heart failure, hypertension, valvular disease and the like.

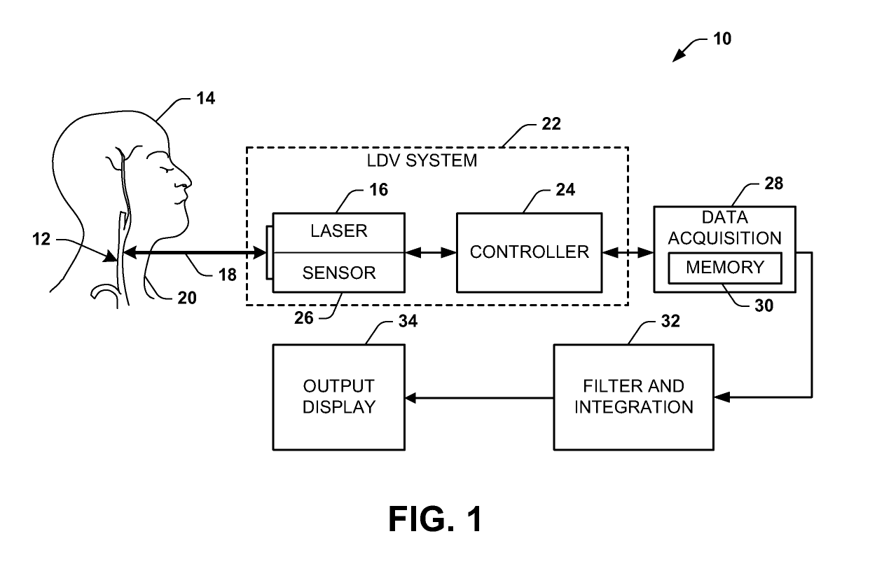

Turning to the figures, FIG. 1 depicts a system that is configured to detect and record the skin velocity and displacement waveforms corresponding to an artery of a subject. In the particular example, of FIG. 1, the system 10 is configured for non-contact measurements of the waveforms for the carotid artery 12 of a patient 14.

A laser 16 provides an optical beam 18 onto a skin surface 20 overlying an artery 12 on a human or animal patient 14. The signal can be obtained through an external data acquisition box in which the system 10 is implemented. In FIG. 1, the system 10 utilizes a laser interferometer, such as can be housed in a laser Doppler vibrometer (LDV) system 22. The LDV system 22, for instance, includes a controller 24 that controls operation of the laser 16 to focus the beam 18 on the skin surface 20 overlying the common carotid artery 12 of the subject in order to measure the skin surface velocity. The laser light reflected from the skin 20, modulated by the skin motion, interferes with a reference beam internal to the vibrometer 22 at an optical sensor 26 of the LDV system. The optical sensor 26 can be implemented as one or more photodiodes that convert the interfering signals to a corresponding electrical sensor signal. The signal out of the optical sensor 26 is processed to extract a physiological signal that is indicative of the skin motion.

The laser output from the optical sensor device focused on the skin can provide information about the velocity and displacement of the underlying artery with minimal noise. Since skin layers are strongly forward scattering media at a red laser wavelength (about 633 nm) 16, they redirect a substantial portion of the laser beam back along its axis towards the optical sensor 26. Such scattered light is detected by the optical sensor 26 and demodulated to provide information on the lateral displacement and velocity of the vessels based on which an arterial blood pressure waveform can be determined. Any hair follicles that exist buried within the skin tissue layers will contribute to some extent to the absorption and scattering of the light and to the physiological noise present in the tissue. Accordingly, the hair can be removed. Alternatively or additionally, a film or sheet of reflective and flexible material can be applied on the skin to reduce noise and enhance the reflection of the incident beam 18.

The output signal corresponding to skin motion can be obtained by a data acquisition block 28 that is configured to sample the skin motion signal. For instance, the output signal corresponding to the skin motion can be represented as an output analog voltage signal, such as corresponding to skin velocity (and/or displacement). The analog voltage signal can be converted to a corresponding digital representation and stored in memory 30 for subsequent processing.

A filter/integration block 32 is configured to filter and integrate the skin velocity data to generate an output waveform corresponding to skin displacement, which can be displayed via an output device 34. For example, the filter 32 can be implemented as a high-pass filter to eliminate DC noise components, such as can be implemented in machine readable instructions, hardware or a combination of hardware and machine readable instructions. Similarly, the integration of the filtered signal can also be implemented in machine readable instructions, hardware or hardware and machine readable instructions. The output device 34 or other components (not shown) can also perform additional processing on the displacement waveform or other measured data to generate an output representing one or more desired physiological conditions. As described herein, the system 10 can provide the output(s) so that variations in the physiological condition(s) over time can be continuously monitored.

By way of example, the skin velocity and displacement waveforms obtained from the optical sensor can be monitored in real-time and recorded for subsequent analysis of the subject's physiological condition. The laser Doppler vibrometer 22 can be compact and portable and capable of measuring skin velocities, for example, ranging from 0.05 um/s to 100 mm/s vibrating at frequencies from 0.5 Hz to 22 kHz. The detection capabilities of the optical sensor 26 can exceed the requirements for monitoring the physiological signals of interest in terms of both sensitivity and bandwidth, but will depend on the quality of the light signal reflected from the skin responsive to the incident beam.

Normal heart rates typically range from 60 to 100 bpm, and respiration rates between 10 and 20 breaths per minute are considered normal, both of which may fluctuate with the level of physical activity, illness, injury, and emotions. The extreme range of heart rates in humans is 0-300 bpm, corresponding to a frequency response of 0-5 Hz. The human arterial blood pressure has a range from 30-300 mmHg, corresponding to a frequency response of 0-100 Hz, which are both well within the capabilities of the system 10. The velocity of the arterial wall in an extreme case of an arterial pressure of 300 mmHg is 40 mm/s, which is also within the velocity detection range of the system 10.

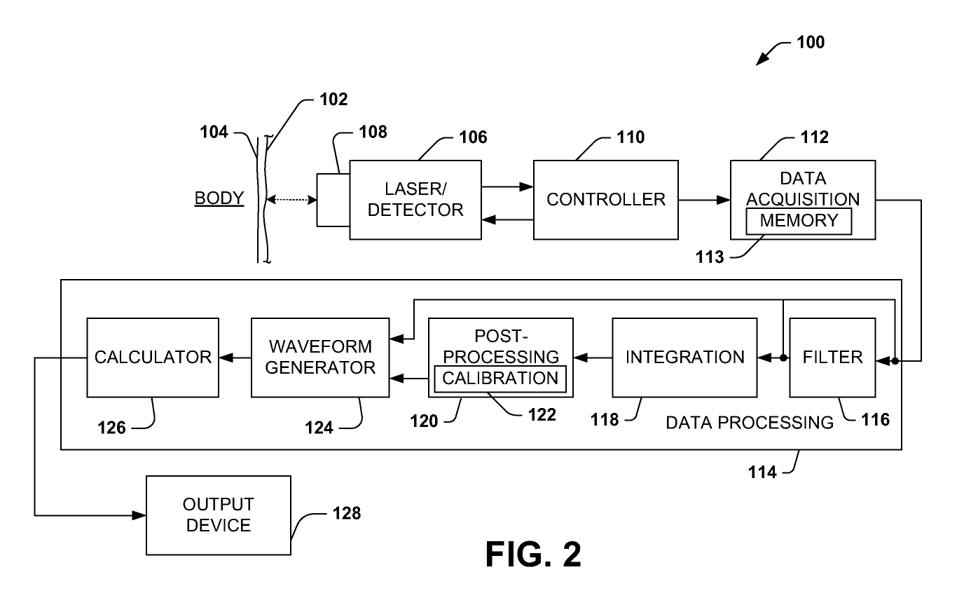

FIG. 2 depicts an example of a system that can be utilized to measure one or more physiological signals of a patient in a non-invasive manner. The system 100 interrogates a surface 102 of a patient's body, such as the skin, which is an overlying relation with an artery (e.g., any of the carotid, facial, temporal, brachial, radial, femoral, popliteal, posterior tibialis, and dorsalis pedis arteries) 104. A reflective material (e.g., a thin film, a sheet or coating) can be positioned on the skin overlying the selected artery 104 to increase the reflectivity at the surface 102.

The system 100 includes a laser/detector 106 that includes a laser configured to provide an optical signal at a desired wavelength (e.g., about 630-700 nm). An optical interface 108 is optically coupled between the laser/detector 106 and the surface 102. The optical interface 108 is configured for providing the optical beam to the surface 102 in a non-contact manner. For example, the optical interface 108 can include an arrangement of optical lenses, an optical fiber that extends from the laser 106 to terminate in a light emitting end that is positioned adjacent to (but not in contact with) the body surface 102. The light emitting end of the optical interface 108 can be positioned within a predetermined distance indicated at “d” from the body surface 102. It will be appreciated that in many embodiments the entire system 100 is positioned in a non-contact relationship with the patient. In another embodiment (e.g., a semi-contact system and method), the laser/detector 106 and/or optical interface 108 can be held in contact or otherwise fixed relative to the patient's body, such as to minimize problems associated with patient movement.

The optical beam is directed at the surface 102 and reflects back at least a portion of the emitted light to the optical interface 108 where it is retrieved by a detector portion of the laser/detector 106. The received optical signal can be compared relative to a corresponding reference beam generated by the laser. The laser/detector 106 can be configured as comprising an interferometer that can superimpose the respective beams together to provide a corresponding pattern that is determined by the phase difference between the respective beams. This difference signal can generate a corresponding analog output electrical signal to the controller 110 representative of the skin velocity at the location where the incident beam is applied.

The laser/detector 106 and controller 110 can be implemented as part of a laser Doppler vibrometer, such as described with respect to FIG. 1. The controller 110 can supply power to the laser as well as perform demodulation and filtering methods to provide the corresponding output signal representative of the velocity at the surface 102. The invention is not limited by a specific laser wavelength used in the interferometer of laser/detector 106. For instance, a variety of interferometer designs can be utilized, such as including a Michelson, Mach-Zhender or other interferometer configuration. The output of the controller 110 thus can provide an electrical output signal (e.g., an analog voltage) corresponding to the skin velocity.

A data acquisition block 112 receives the output waveform and stores or buffers in memory 113 a corresponding sample thereof continuously over time. For instance, the data acquisition block 112 can provide the acquired waveform as an analog waveform that has been normalized with respect to a voltage reference. Alternatively, the data acquisition block 112 can implement analog-to-digital conversion and sampling of the output signal and, in turn, provide a corresponding digitized output signal.

The data acquisition block 112 provides the output waveform to a data processing block (e.g., a processor) 114. The data processing block 114 is configured for processing the output waveform to generate a corresponding waveform that represents one or more physiological conditions. The data processing block 114 can also calculate one or more values that relate to the physiological condition. The data processing block 114 can be implemented as hardware (e.g., in a microprocessor, an application specific integrated circuit (ASIC)), machine readable instructions (e.g., executable instructions running on a processor or other processor-based device) or a combination thereof, which can be configured to perform the functions described herein.

In the example of FIG. 2, the data processing block 114 includes a filter 116 that filters the output waveform (e.g., corresponding to velocity data) to provide filtered data. For example, the filter 116 can be implemented as a high or band pass Butterworth filter, although other filter topologies can also be utilized. The filtered data is provided to an integration block 118. The integration block 118 in turn integrates the filtered waveform data with respect to time to provide a corresponding signal representing displacement. Thus, where the output waveform from the data acquisition block represents skin velocity, the output of integration component 118 can represent skin displacement.

The displacement waveform can be provided to a corresponding post-processing block 120. The post processing block 120 is configured to perform the calibration (indicated at 122) to provide a calibrated displacement waveform that represents a physiological condition, such as corresponding blood pressure waveform. For instance, the calibration 122 can be implemented for adjusting the amplitude, phase, or a combination of amplitude and phase of the displacement waveform.

As one example, the displacement waveform can be calibrated to the “gold-standard,” invasive arterial blood pressure waveform, in order to produce the absolute blood pressure waveforms. The calibration 122 can be performed, for instance, as part of a start-up procedure for a given patient when the device is positioned for operation. The results of the calibration can in turn be utilized to set one or more calibration parameters applied by the calibration component 122 for adjusting the displacement waveform to represent a continuous measurement of a patient's blood pressure.

By way of further example, several calibration techniques may be considered for determining a calibration factor, which can be implemented on a patient-by-patient basis. For instance, a patient may initially have standard cuff measurements taken as is done to calibrate the pressure values for the commercial SphygmoCor device, which is available from AtCor Medical Pty Ltd. of Itasca, Ill. A calibration factor could then be calculated based on the systolic and diastolic blood pressures taken and applied to the skin displacement waveform obtained from the present invention. In patients with existing intra-arterial catheters monitoring the blood pressure waveform, the same calibration methods described above could be used with the arterial blood pressure waveform as the standard against which to calibrate the skin displacement waveform.

Additionally, the diastolic decay and systolic growth portions of the blood pressure waveform can be used for further calibration. A ratio of systolic/diastolic pressures yields a unit-less figure of merit to compare the skin displacement and arterial blood pressure signals. This calibration technique would be particularly useful in ambulatory situations, or in patients undergoing sleep studies, where the initial standard cuff measurements cannot be tolerated to calibrate the system 100. Typically, calibration of the system 100 may involve contact or invasive methods for obtaining pressure values. Calibration to actual pressure values is not necessary, however, since information on the blood pulse waveform indicative of cardiac condition will be obtained regardless of the absolute signal amplitude.

The calibrated displacement waveform data can be provided to a corresponding waveform generator 124, which can generate one or more output signals for rendering a graphical representation of the blood pressure waveform on an output device 128. The waveform generator 124 can also be utilized to generate a corresponding skin velocity waveform, such as can be based upon the filtered signal from the filter 116 or from the pre-filtered waveform provided from the data acquisition block 112.

The data processing block 114 can also include a calculator 126 that is programmed and/or configured to compute a value for one or more desired physiological conditions based on the detected and derived physiological signals. As described herein, the computed values can provide an indication of a variety of physiological conditions such as may include, but are not limited to, systolic blood pressure, diastolic blood pressure, respiration rate, heart rate, as well as other conditions associated with the flow of blood through the body and/or cardiac health.

The calculator 126 can also be programmed with methods to compute or derive an indication of other health-related parameters. For example, the calculator 126 can utilize the blood pressure waveform to compute stroke volume, cardiac output, patient volume signals (e.g., an indication of hydration), vascular resistance, shock status, and arterial disease (e.g., an indication of carotid obstruction). Thus, it can serve as a diagnostic tool. The calculator 126 can also employ methods programmed to measure (indirectly) endothelial function and arterial stiffness, which have been correlated with clinical outcomes. Thus, the system 100 can be implemented as part of a screening tool for coronary and cerebrovascular disease.

The output device 128 can include a display that may display the computed values from the calculator 126, one or more waveforms generated by the waveform generator 124 or a combination of calculated values and waveforms. The output device 128 may provide the output locally for display on the device. It will be appreciated that the system 100 (including the laser/detector 106, controller 110, data acquisition block 112), the data processing block and the output device 128 can be integrated within a housing or enclosure, such that the system can be considered portable. As a result of such portability, the system 100 can be utilized in a variety of care settings including in hospitals, medical offices, home health care and the like.

As an alternative example, the processing block 114 can be remotely located (e.g., via a wired, optical or wireless communications link) from the laser/detector 106, controller 110, data acquisition block 112, such that the acquired data is communicated to the processing block (e.g., a computer or processing based unit) for performing desired computations and generating waveforms and other information for display. It will be appreciated that functionality described herein could be distributed between the unit at the patient and a remote unit.

As a further example, the output device 128 may include data communications interface for communicating the corresponding output(s) to a remote system (not shown), such as a central monitoring system that may collect data from any number of systems 100. Such data communications with the central monitoring system can be wireless or be implemented through a physical connection to a network. When the data is being communicated remotely, the output device 128 can packetize the data with a header identifying the system 100 uniquely with respect to other systems being utilized for other patients.

As another alternative example, the output device 128 can provide an interface for communicating the information from the system 100 to integrate this information with information from one or more other types of sensors that may be monitoring other patient conditions such as electrograms, patient temperature or the like. In view of the examples described herein, a variety of implementations and uses for the system 100 can be made.

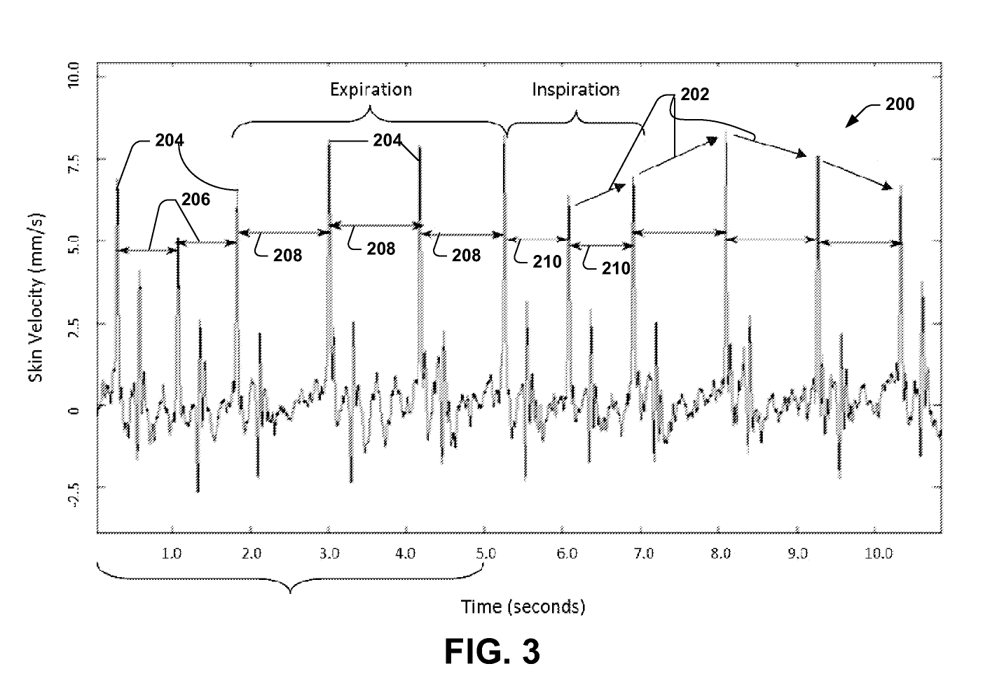

FIG. 3 depicts an example of a skin velocity waveform, such as can be obtained from systems and methods shown and described herein. When utilizing the system for measurement at the carotid artery, for example, the measured signal also contains low frequency motion associated with respiration. Due to the location of the carotid artery, the vibrations of the trachea can also be observed by following the envelope of the amplitude peaks, demonstrated at 202.

The heart rate and respiratory rate for the signal depicted in FIG. 3 are approximately 60 beats per minute and 12 breaths per minute, respectively. The heart rate can be easily determined (e.g., by a calculator function 126 of FIG. 2) by counting the number of easily distinguishable peaks 204 in the skin velocity waveform 200 in a given time interval 206, and multiplying by the appropriate factor (in this example, 12) to convert the reading into beats per minute. Once the heart rate has been determined from the skin velocity waveform, various systolic time intervals can be determined. For example, three basic systolic time intervals are the pre-ejection period (PEP), left ventricular ejection time (LVET) and total electromechanical systole (QS2). Linear relationships between heart rate (HR) and the duration of the systolic phases of the left ventricle (LV) have been derived by observation. The following equations can thus been utilized to predict the durations of the systolic time intervals for normal patient observations based on the heart rate determined from the skin velocity waveform 200 (see, e.g., FIG. 3):

PEP=−0.0004*HR+0.126 (1)

LVET=−0.0016*HR+0.394 (2)

QS2=−0.020*HR+0.522 (3)
See, e.g., Lewis et al, Circulation v.56, 1977; Lewis et al. Am J of Cardiology, v.37, 1976; Weissler et al, Circulation v.37 1968; Hamosh and Cohn, Circulation v.45 1972

Information about the systolic time intervals is useful in assessing a patient's cardiac health, and can help to identify disease states when measurements deviate from the regression equations listed above. Systolic time intervals can be used to assess disease states including left ventricular failure, myocardial infarction, coronary artery disease, and valve disorders. The dicrotic notch indicates the timing of the closure of the aortic valve, marking the end of left ventricular ejection, and can be located on the blood pressure waveform to predict the systolic time interval as a function of the heart rate through the equations listed above. The regression equations are expected to deviate in patients with cardiac dysfunction.

The respiration of the subject can be demonstrated by the amplitude envelope of the peaks. The subject having the example waveform of FIG. 3, had a pronounced sinus arrhythmia, so the respiration of the patient could also be appreciated between the skin velocity peaks since the heart rate of the patient increases during inspiration, and the heart rate decreases during expiration. Thus, as demonstrated by the arrows 208 and 210 in FIG. 3, the distance between the peaks during expiration, indicated at 208, will be longer than those during inspiration, indicated at 210.

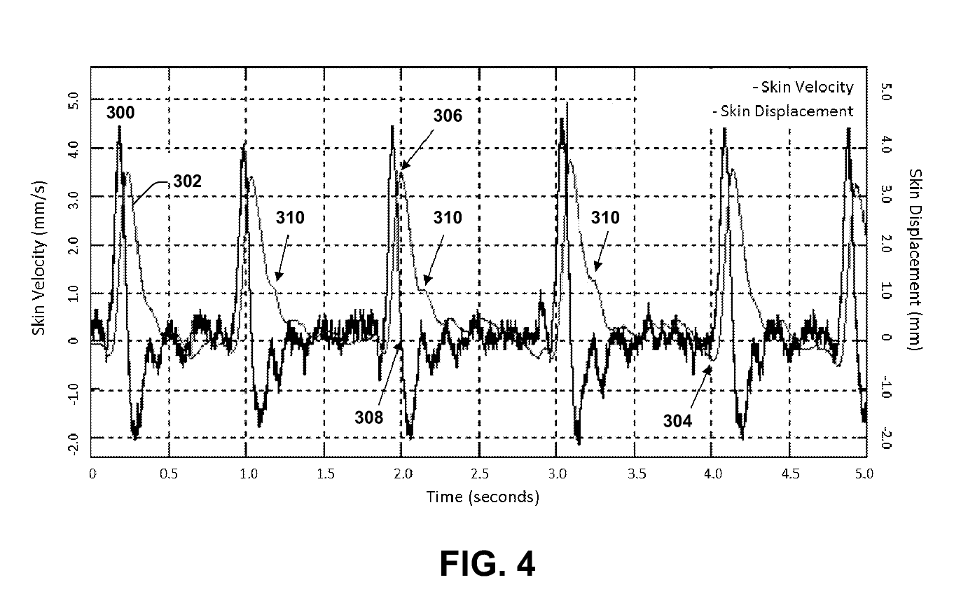

Once the pulsatile skin velocity waveform is obtained, a computer or other processing device (e.g., data processing block 114 of FIG. 2) can derive a highly accurate representation of the blood pressure waveform. Examples of various waveforms are shown in FIGS. 4-6. Integration of the skin velocity signal provides the skin displacement waveform, which can be adjusted (or calibrated) to provide a blood pressure waveform that is commensurate with clinical blood pressure waveforms that can be obtained invasively.

FIG. 4 demonstrates a skin velocity signal 300 for an artery (e.g., as detected by the LDV of FIG. 1 or 2) and a corresponding skin displacement waveform 302. The minima 304 and maxima 306 in the calculated displacement signal correspond to zero crossings in the measured velocity waveform 308. Peak systole 306 is clearly visible on the skin displacement waveform. The dicrotic notch 310, corresponding to the closing of the aortic valve, can also be easily identified in the displacement waveform 302 and velocity waveform 300.

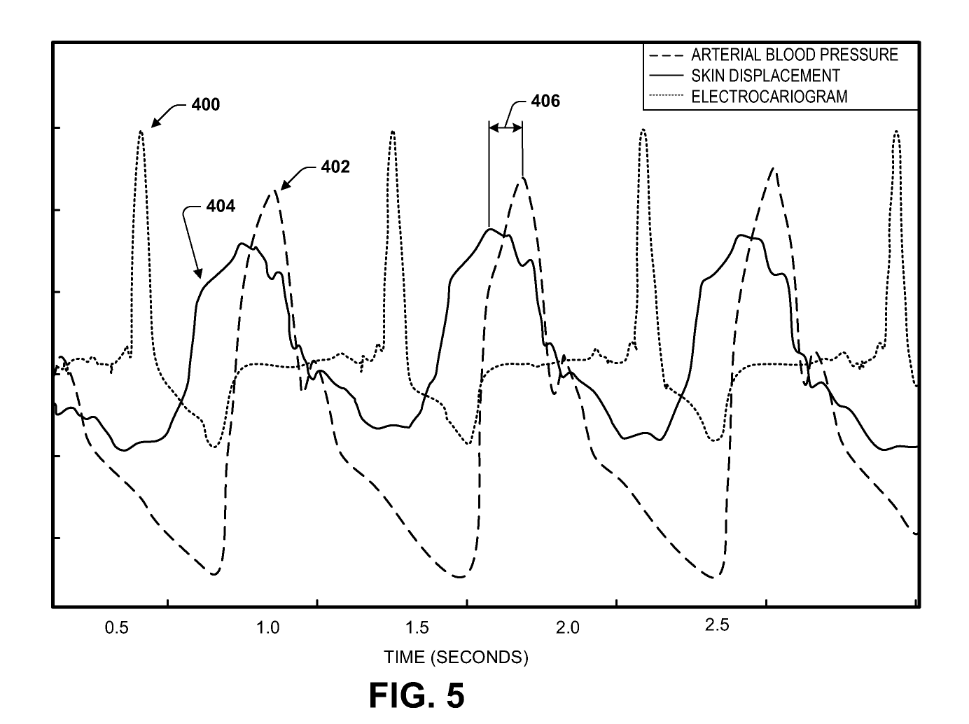

FIG. 5 demonstrates a qualitative comparison of three waveforms obtained for a given time period from simultaneous measurements. Specifically, FIG. 5 depicts an electrocardiogram 400, arterial blood pressure waveform 402, and LDV skin displacement waveform 404 obtained for a given patient. Each of the waveforms 400, 402 and 404 presented in FIG. 5 were scaled and shifted in order to view them all on the same time axis. Thus, although the y-axis amplitude is not significant, relevant qualitative relationships between the waveforms can be observed. For the particular example waveforms in FIG. 5, it is to be understood that the skin velocity (and thus displacement 404) waveforms were obtained for the carotid artery using the LDV, whereas the invasive arterial blood pressure waveform 402 was taken from the radial artery, which explains the slight time difference, indicated at 406, between the peaks of both waveforms 402 and 404.

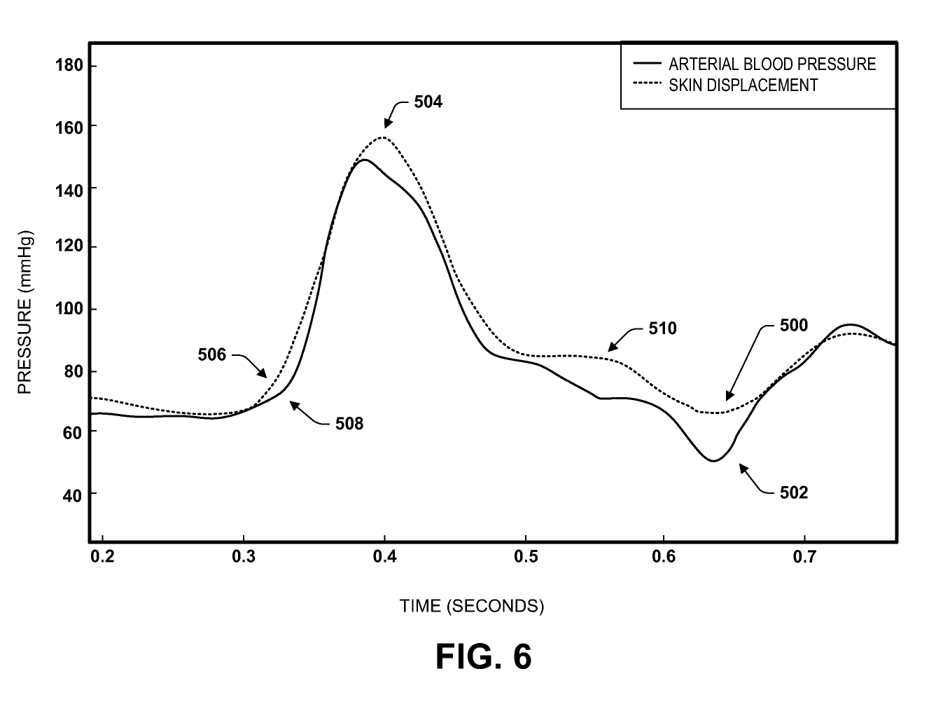

FIG. 6 depicts a qualitative comparison of an arterial blood pressure waveform 500 and a skin displacement waveform 502. One cardiac cycle of the carotid skin displacement and radial artery blood pressure waveforms were extracted from the same patient to produce the information demonstrated in the example of FIG. 6. The signal amplitudes were scaled, and the skin displacement waveform was time shifted to align with the timing of peak systole 504 from the invasive arterial signal 500. As expected, the initial upward slope of the radial artery signal, indicated at 506, is steeper than that of the carotid indicated at 508, (since it lies more distal to the heart). Additionally, the dicrotic notch 510 is smoother. The carotid artery signal in general presents more detail than the radial artery.

Those skilled in the art will also appreciate that portions of the invention may be embodied as a method, data processing system, or computer program product. Accordingly, these portions of the present invention may take the form of an entirely hardware embodiment, an entirely software or firmware embodiment, or an embodiment combining software and hardware. Furthermore, portions of the invention may be a computer program product on a computer-usable storage medium having computer readable instructions on the medium. Any suitable computer-readable medium may be utilized including, but not limited to, static and dynamic storage devices, hard disks, optical storage devices, and magnetic storage devices.

Certain embodiments of the invention have also been described herein with reference to block illustrations of methods, systems, and computer program products. It will be understood that blocks of the illustrations, and combinations of blocks in the illustrations, can be implemented by computer-executable instructions. These computer-executable instructions may be provided to one or more processor of a general purpose computer, special purpose computer, or other programmable data processing apparatus (or a combination of devices and circuits) to produce a machine, such that the instructions, which execute via the processor, implement the functions specified in the block or blocks.

These computer-executable instructions may also be stored in computer-readable memory that can direct a computer or other programmable data processing apparatus to function in a particular manner, such that the instructions stored in the computer-readable memory result in an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

In view of the foregoing, those skilled in the art may appreciate various changes in the details, materials, steps and arrangement of parts. For example, it may be desirable to utilize a fiber optic means for directing and/or detecting the laser beams of interest. Due to the motion of a patient's skin surface, which is not directly related to the measurement of the biological signal of interest, e.g., blood pressure waveform, an adaptive focus may be utilized to maintain the interrogating laser beam on the desired measurement area, such as the carotid artery.

As another example, an embodiment of the invention can be utilized in cardiac and electrophysiology procedures that require non-invasive, in addition to invasive monitoring of blood pressure. For instance, the presence of the sphygmomanometer and its cables can be a logistical problem to imaging equipment and work flow in a catheterization laboratory, such as in situations when performing procedures from the radial artery or subclavian.

Additionally, while some embodiments of the invention have been described herein for use non-invasively, it will be appreciated that other embodiments of the invention can be used invasively. For example, an apparatus employing an optical fiber can be applied in a non-contact manner in conjunction with endoscopic procedures, such as to measure pulmonary artery displacement (hence pressure waveforms) in thoracic surgery procedures.

What have been described above are examples and embodiments of the invention. It is, of course, not possible to describe every conceivable combination of components or methodologies for purposes of describing the invention, but one of ordinary skill in the art will recognize that many further combinations and permutations of the present invention are possible. Accordingly, the invention is intended to embrace all such alterations, modifications and variations that fall within the scope of the appended claims. In the claims, unless otherwise indicated, the article “a” is to refer to “one or more than one.”

What is claimed is:

1. A system for non-contact measurements of a physiological condition, comprising:
a laser configured to optically interrogate a surface of a patient's body with an optical signal;
a detector configured to receive reflected light from the surface in response to the optical signal;
a controller configured to determine velocity data representing a velocity of the surface based on the optical signal and the reflected light; and
a processor configured to compute a corresponding displacement waveform representing displacement of the surface based on the velocity data, the corresponding displacement waveform representing the physiological condition.

2. The system of claim 1, further comprising an optical interface between the laser and the surface, the optical interface being spaced apart from the surface and configured for directing the optical beam to the surface.

3. The system of claim 2, wherein the optical interface comprises an optical fiber.

4. The system of claim 1, wherein the processor further comprises:
a filter configured to filter the velocity data and provide a filtered waveform signal; and
an integrator configured to integrate the velocity data to provide an integrated output signal based on which the corresponding displacement waveform is computed.

5. The system of claim 4, wherein the processor further comprises a post-processing component configured to adjust at least one of amplitude and phase of the integrated output signal to provide the corresponding displacement waveform.

6. The system of claim 1, wherein the corresponding displacement waveform represents a continuous blood pressure waveform for the patient.

7. The system of claim 1, wherein the surface where the optical signal is directed overlies an artery.

8. The system of claim 1 implemented as a portable unit wherein the laser, the detector and the processor reside in a housing.

9. The system of claim 1, wherein the physiological condition is at least one of blood pressure, respiration rate and heart rate.

10. The system of claim 1, further comprising an output device configured to present a visual representation of the corresponding displacement waveform representing the physiological condition.

11. The system of claim 1, wherein the detector further comprises an interferometer configured to provide the voltage data based on the reflected optical signal and a reference optical signal and a controller to accept the signal data from the detector to produce a velocity signal.

12. A system for non-contact measurements of a physiological condition, comprising:
a laser configured to emit an optical signal to a surface of a patient's body;
an optical detector configured to receive a reflected optical signal from the surface in response to the emitted optical beam;
a controller configured to determine velocity data representing a velocity of the surface based on the detected signal;
a data acquisition component configured to collect the velocity data;
an integrator configured to integrated the velocity data with respect to time; and
a waveform generator configured to generate a displacement waveform representing displacement of the surface based on the velocity data, the displacement waveform corresponding to the physiological condition.

13. The system of claim 12, further comprising a filter configured to filter the velocity data and provide filtered velocity data, the integrator integrating the filtered velocity data to provide an integrated output signal based on which the corresponding displacement waveform is computed.

14. The system of claim 12, further comprising an optical interface between the laser and the surface, the optical interface being spaced apart from the surface and arranged for directing the optical signal to the surface.

15. The system of claim 14, wherein the optical interface comprises an optical fiber.

16. The system of claim 12, further comprising a portable housing, the laser, the optical detector and the controller residing in the portable housing.

17. The system of claim 12, wherein the physiological condition is at least one of blood pressure, respiration rate and heart rate.

18. The system of claim 12, further comprising an output device configured to present a visual representation of the displacement waveform representing the physiological condition.

19. The system of claim 12, further comprising a post-processing block configured to perform calibration to set operating parameters for adjusting at least one of amplitude and phase of the displacement waveform.

20. The system of claim 12, wherein the laser/detector further comprises an interferometer configured to determine the skin motion data based on the reflected optical signal an a reference optical signal and a controller to convert the detected signal into calibrated velocity data.
